(19) United States
(12) Patent Application Publication
(10) Pub. No.: US 2007/0148765 A1
(43) Pub. Date: Jun. 28, 2007

(54) Preservative-containing virus formulations

(76) Inventors: Robert K. Evans, Souderton, PA (US); Daniel H. Yin, Schwenksville, PA (US)

Correspondence Address: MERCK AND CO., INC, P O BOX 2000, RAHWAY, NJ, 07065-0907, US

(21) Appl. No.: 10/578,955

(22) PCT Filed: Nov. 18, 2004
(86) PCT No.: PCT/US04/38670

§ 371 Date: May 10, 2006

Related U.S. Application Data
(60) Provisional application No. 60/523,479, filed on Nov. 19, 2003.

(51) Int. Cl. C12Q 1/68; C12P 21/06; C12N 15/00

(52) U.S. Cl. 435/320.1; 435/69.1; 435/6

(57) ABSTRACT

The preservation of live viral vaccines is disclosed. These liquid formulations comprise a live virus and a preservative, namely chlorobutanol. The preserved, live virus formulations of the present invention are (1) suitable for a vaccine or gene therapy product with a multi-dose image; (2) compatible with parenteral administration; and (3) are stable for extended periods of time with negligible loss of activity.

### CROSS-REFERENCE TO RELATED APPLICATIONS

This application claims benefit, under 35 U.S.C. §119(e), to U.S. provisional application 60/523,479 filed Nov. 19, 2003.

### STATEMENT REGARDING FEDERALLY-SPONSORED R&D

Not Applicable

### REFERENCE TO MICROFICHE APPENDIX

Not Applicable

### FIELD OF THE INVENTION

The present invention relates to liquid formulations comprising a live virus and a preservative, as well as related pharmaceutical products for use in vaccine and/or gene therapy applications and associated methods of preparing these formulations. The preserved, live virus formulations of the present invention are (1) suitable for a vaccine or gene therapy product with a multi-dose image; (2) compatible with parenteral administration; and (3) are stable for extended periods of time with negligible loss of activity. Exemplified herein is a live adenovirus formulation comprising chlorobutanol, which possesses the above-mentioned characteristics.

### BACKGROUND OF THE INVENTION

A specific challenge, especially in the field of live virus vaccines, is to generate a multi-dose liquid live virus formulation which shows both viral stability as well as antimicrobial effectiveness. Because of the potential of recombinant adenovirus vectors in the fields of vaccines and gene therapy, there is a specific need for development of an adenoviral-based vaccine which has a multi-dose image due at least in part by the addition of a preservative that does not negatively effect viral stability while also inhibit the growth of microorganisms that may be introduced from repeatedly withdrawing individual doses.

Recombinant live adenovirus vectors have been proposed for use as a HIV vaccine. Regions of the world most in need of an HIV vaccine are in the developing world, areas where large vaccination campaigns would be an effective strategy for control of HIV. To best support such campaigns a multi-dose image of the vaccine would make more practical and economical sense than a single-dose vial image.

Kowalski et al. (1998, Am. J. Ophthalmology 126(6): 835-836) and Romanowski et al. (1999, Am. J. Ophthalmology 128(2): 239-240) showed data indicating that antimicrobial preservatives significantly reduced the stability of adenovirus and herpes simples virus, respectively, in various ophthalmic solutions. The authors were investigating the ability of these viruses to contaminate ophthalmic solutions in the office setting, possibly serving as a source of unwelcome patient infection.

WO 01/66137 discloses virus formulations that may comprise a buffer, a sugar, a salt, a divalent cation, a non-ionic detergent, as well as a free radical scavenger and/or chelating agent to inhibit free radical oxidation.

To date, the inventors are not aware of any examples of a commercial live virus vaccine containing a preservative. Multi-dose vaccine products without preservatives must presently be discarded at the end of each immunization session or at the end of six hours, whichever comes first. Therefore, there has been and remains a need for a stable liquid viral formulation which is suitable for a multi-dose live virus based vaccine, which may be used in subsequent immunization sessions. The present invention meets this need by disclosing liquid formulations comprising a live virus and a preservative for use in vaccine and/or gene therapy applications. The preserved, live virus formulations of the present invention are suitable for filling in a multi-dose vaccine vial or container, is compatible with parenteral administration, and retains stability for extended periods of time at 2-8° C. with negligible loss of activity when compared to the same formulation minus preservative.

### SUMMARY OF THE INVENTION

The present invention relates to live, preserved and stable virus formulations and related pharmaceutical products for use in gene therapy and/or vaccine applications and methods of preserving such stabilized formulations. The stabilized virus formulations of the present invention contain a preservative, which provides for multi-dose formulations. The virus formulations of the present invention are (1) suitable for a vaccine or gene therapy product with a multi-dose image; (2) compatible with parenteral administration; and (3) are stable for extended periods of time with negligible loss of activity. Possible preservatives approved for use in injectable drugs which may be compatible with the live virus formulation while having regulatory acceptance include but are not necessarily limited to chlorobutanol, m-cresol, methylparaben, propylparaben, 2-phenoxyethanol, benzethonium chloride, benzalkonium chloride, benzoic acid, benzyl alcohol, phenol, thimerosal and phenylmercuric nitrate. Such live viral vaccines are contemplated as part of the present invention. In a further embodiment of the present invention, the live virus formulation is a formulation that contains the preservative chlorobutanol at an effective concentration to promote antimicrobial activity.

The present invention further relates to a preserved and stable virus formulation and related pharmaceutical product for use in gene therapy and/or vaccine, and methods of preserving such stabilized formulations, wherein the live virus is adenovirus or a recombinant form of adenovirus, such as a replication deficient adenovirus as known in the vaccine and gene therapy art and as discussed infra. Again, possible preservatives approved for use in injectable drugs which may be compatible with the live virus formulation while have regulatory acceptance include but are not necessarily limited to chlorobutanol, m-cresol, methylparaben, propylparaben, 2-phenoxyethanol, benzethonium chloride, benzalkonium chloride, benzoic acid, benzyl alcohol, phenol, thimerosal and phenylmercuric nitrate. Such live adenoviral vaccines are contemplated as part of the present invention. In a preferred embodiment of the present invention, the live adenovirus formulation is a formulation which contains the preservative chlorobutanol at a biologically effective concentration to promote antimicrobial activity.

Formulation candidates to preserve adenovirus are liquid adenovirus formulations showing improved stability when stored in about the 2-8° C. range while also being compatible with parenteral administration. A preferred family of formulations may comprise a buffer, a sugar, a salt, a divalent cation, a non-ionic detergent, as well as a free radical scavenger and/or chelating agent to inhibit free radical oxidation. This family of stabilizing virus formulations are disclosed within PCT International Application PCT/US01/07194 (International Publication No. WO 01/66137). To this end, the present invention relates to live adenovirus formulations, and methods of preserving such stabilized formulations, both as a proposed single dose or a multi-dose image, as well as single-dose or multi-dose filled vaccine vials, which comprise a live adenovirus and a biologically effective concentration of chlorobutanol. As used herein, a “biologically effective concentration” or “effective concentration” of chlorobutanol is a concentration of chlorobutanol within the viral formulation of interest which imparts an antimicrobial effect above and beyond that of the same formulation lacking chlorobutanol while remaining soluble within the respective formulation at a physiologically relevant temperature, while also possessing additional characteristics described herein. Therefore, while the range of chlorobutanol in exemplified adenovirus formulations disclosed herein is from 0.25% to 0.6% (w/v), this chlorobutanol concentration range is in no way forwarded as a limitation, but instead as a guide to show the artisan that any concentration of chlorobutanol which both promotes antimicrobial activity and remains soluble within the formulation is useful and is part of the core teaching of the present invention. These formulations may further comprise at least one inhibitor of free radical oxidation (including but not limited to EDTA, ethanol, histidine or any multiple combination thereof); may contain various amounts of a buffer, a cryoprotectant, a salt, a divalent cation, and a non-ionic detergent; and may have a final formulation concentration of live adenovirus in the range from about 1×10⁷ vp/mL to about 1×10¹³ vp/mL; and/or a total formulation osmolarity in a range from about 200 mOs/L to about 800 mOs/L.

The present invention also relates to a method of preserving a live adenovirus formulation, as a single dose or multi-dose filling, which comprises adding chlorobutanol to the formulations described herein, such that addition of chlorobutanol effectively preserves the adenovirus while maintaining stability of the adenovirus for an extended period of time with negligible loss of virus potency. The formulation of the present invention, with addition of chlorobutanol at concentrations as disclosed herein, provide adequate stability for at least 1-2 years when stored at 2-8° C.

### BRIEF DESCRIPTION OF THE DRAWINGS

FIG. 1 shows the log loss of adenovirus infectivity at 37° C. for 14 weeks vs. −70° C. storage for A195 alone [●]; 0.5% chlorobutanol (CB) [□]; 0.18% methylparaben (MP) and 0.02% propylparaben (PP) [∘]; 0.5% 2-phenoxyethanol (PE) [▴]; 0.2% benzoic acid (BZ) [⋄]. The infectivity of adenovirus was measured using QPA with assay variation of +/−0.15 logs. From this short-term study under accelerated conditions, chlorobutanol and benzoic acid show no significant effects on the stability of adenovirus (see Table 4)

FIG. 2A-D show the effect of chlorobutanol concentration on the stability of MRKAd5gag formulated in A195 buffer. (A) 37° C., pH7.4; (B) 30° C., pH7.4; (C) 25° C., pH7.4; (D) 20° C., pH7.4.

FIG. 3A-D show the stability of MRKAd5gag was formulated in A195 buffer containing 0.5% (w/v) chlorobutanol at pH 6.0, 6,8 and 7.4, respectively and stored at 37° C. (A); 30° C. (B); 25° C. (C); and, 20° C. (D).

FIG. 4 shows an overlap fitting the stability of adenovirus in A195 over the stability data of A195 in 0.5% chlorobutanol stored at 37, 30, 25, and 20° C. The Arrhenius plot indicates that the projected loss of adenovirus infectivity in A195 is ≦0.1 logs after 2 years of 2-8° C. storage, with or without the presence of chlorobutanol. (●) Previous Arrhenius analysis of adenovirus stability data (in A195 buffer, storage temperature: 37, 30, 25, 15, 5° C.); (□) Arrhenius analysis of accelerated adenovirus stability data (in A195+0.5% chlorobutanol, pH 6.8, at 37, 30, 25, 20° C.). The data suggests that a long-term stability will be similar to the A195 formulation.

FIG. 5 shows the Arrhenius plot of chlorobutanol degradation rate constants.

FIG. 6 shows the pH dependent Arrhenius factor as calculated in FIG. 5.

FIG. 7 shows calculated pH changes in A195 buffer due to chlorobutanol degradation.

### DETAILED DESCRIPTION OF THE INVENTION

The present invention relates to live, stable and preserved virus formulations and related pharmaceutical products for use in gene therapy and/or vaccine applications and methods of preserving such stabilized formulations. The stabilized virus formulations of the present invention contain a preservative, which allows for multi-dose formulations in a commercial setting. The virus formulations of the present invention are (1) suitable for a vaccine or gene therapy product with a multi-dose image; (2) compatible with parenteral administration; and (3) are stable for extended periods of time with negligible loss of activity.

To develop an injectable multi-dose live virus vaccine comprising a preservative-containing formulation, consideration should be given to several factors when choosing suitable level(s) of preservative(s) and designing tests to establish efficacy of a preservative system. More specifically, the formulation of the present invention will be (1) nontoxic to the recipient in the recommended dose; (2) compatible with the specific substances in the product within the shelf life, an example being that the preservative is soluble within the respective formulation; (3) have minimal effects on vaccine potency; and, (4) possess demonstrable antimicrobial effectiveness. For commercial applications, it will additionally be useful that the formulation possess scale-up capability. There are presently thirteen FDA approved preservatives that have been used in injectable drugs. Possible preservatives approved for use in injectable drugs which may be compatible with the live virus formulation while having regulatory acceptance include but are not necessarily limited to chlorobutanol, m-cresol, methylparaben, propylparaben, 2-phenoxyethanol, benzethonium chloride, benzalkonium chloride, benzoic acid, benzyl alcohol, phenol, thimerosal and phenylmercuric nitrate. Such live viral vaccines are contemplated as part of the present invention.

Regions of the world most in need of an HIV vaccine are in the developing world, areas where large vaccination campaigns would be an effective strategy for control of HIV. To best support such campaigns multi-dose vaccine containers would be useful, both in a practical and economical sense. Presently, the FDA requires that biological products in multiple-dose vials contain a preservative, with only a few exceptions. Vaccine products containing preservatives include vaccines containing benzethonium chloride (anthrax), 2-phenoxyethanol (DTaP, HepA, Lyme, Polio (parenteral)), phenol (Pneumo, Typhoid (parenteral), Vaccinia) and thimerosal (DTaP, DT, Td, HepB, Hib, Influenza, JE, Mening, Pneumo, Rabies). However, there is no historical precedent for a commercial live virus vaccine containing preservatives because of the perceived incompatibility of the preservative with the viral proteins.

Therefore, the present invention specifically relates to a live adenovirus formulation comprising a preservative selected from, but not limited to, chlorobutanol, m-cresol, methylparaben, propylparaben, 2-phenoxyethanol, benzethonium chloride, benzalkonium chloride, benzoic acid, benzyl alcohol, phenol, thimerosal and phenylmercuric nitrate, which is suitable as a multi-dose image, is compatible with parenteral administration and is stable for extended periods of time with negligible loss of activity. As used herein, a “live virus” or “live virus vaccine” is meant to include, but not necessarily be limited to, virulent serotypes of known viruses (e.g., wild type or modified forms of various adenovirus serotypes as discussed infra), live attenuated vaccines (e.g., viral vaccines which are live but non-pathogenic due to reduced virulence, usually by serial passage of the pathogen through cell culture techniques) and a live recombinant vaccine which will contain a gene(s) or portions thereof which encode for a immunogenic protein or peptide which is expressed upon in vivo administration of this recombinant vectored vaccine). An example of the latter can be found throughout the Example sections, where a live recombinant E1-deficient adenovirus vector (MRK5) which contains the open reading frame for HIV gag, pol or nef, respectively, is formulated in A195 formulation buffer in the presence of various preservatives.

The present invention more specifically relates to live adenovirus formulations which comprise the preservative chlorobutanol at a biologically effective concentration and methods of preserving such stabilized formulations. It will be understood that “biologically effective concentration” or “effective concentration” as used herein is defined as a concentration of preservative in the final live viral formulation which promotes the required preservative criteria as noted above, namely (1) being nontoxic to the recipient in the recommended dose; (2) being compatible with the specific substances in the product within the shelf life (again, a specific example being that the preservative is soluble within the respective formulation); (3) having a minimal effect on vaccine potency; and, (4) possessing demonstrable antimicrobial effectiveness. A recommended requirement for commercial purposes, but not a limiting factor for the formulations of the present invention, is an ability for the preserved viral or adenoviral formulation to be amenable to scaled-up production processes. The artisan may test various CB concentrations to choose the concentration optimal for a specific live adenovirus formulation. As noted above, the range of chlorobutanol in exemplified adenovirus formulations disclosed herein is from 0.25% to 0.6% (w/v). However, this chlorobutanol concentration range is in no way forwarded as a limitation, but instead as a guide to show the artisan that any concentration of chlorobutanol which both promotes antimicrobial activity and remains soluble within the formulation is useful and is part of the core teaching of the present invention. The solubility of chlorobutanol in water at 20° C. is 0.8% (w/v) (see Kibbe, 2000, Handbook of Pharmaceutical Excipients, 3rd Ed., pp 126-128). Therefore, the application of chlorobutanol as an antimicrobial preservative is primarily limited by its solubility. In the present invention chlorobutanol was used in aqueous formulations at 2-8° C. The concentration range of CB exemplified in the invention is 0.25-0.6% in A195 (pH 6.0 to 7.4). Because of CB at near saturation in aqueous buffers, the buffers were prepared by diluting a stock solution of CB in ethanol (48%, v/v) into A195 buffer (pH 6.0 to 7.4, no ethanol). Ethanol in the final solution helps to stabilize the solubility of CB in the aqueous buffers at exemplified concentrations. It is exemplified herein that 0.6% CB is compatible with the stability of adenovirus. Presently, 0.4% CB is required to license a killed multi-dose formulation in the United States, with 0.5% CB required in Europe. Presently, according to the CDER Inactive Ingredient Database, the highest concentration for CB in injectables is 0.60%. Therefore, while a commercially preferred range of chlorobutanol in the viral formulations of the present invention may be from about 0.4% to about 0.6% (w/v) and is therefore considered to be part of the present invention; this present invention also including the exemplified range of from about 0.25% to about 0.6% (w/v), and also teaching, and therefore relating to and covering, a range starting from a lowest biologically effective concentration up to the maximum solubility limit of chlorobutanol in the respective viral formulation; a concentration which will surpass 0.6% (w/v) and possibly 0.8% (w/v), depending on the base viral formulation.

Adenoviruses are non-enveloped, icosahedral viruses that have been identified in several avian and mammalian hosts; Horne et al. (1959 J. Mol. Biol. 1:84-86); Horwitz, 1990, In Virology, eds. B. N. Fields and D. M. Knipe, pps. 1679-1721. The first human adenoviruses (Ads) were isolated over four decades ago. Since then, over 100 distinct adenoviral serotypes have been isolated which infect various mammalian species, 51 of which are of human origin; Straus, 1984, In The Adenoviruses, ed. H. Ginsberg, pps. 451-498, New York:Plenus Press; Hierholzer et al. (1988, J. Infect. Dis. 158:804-813); Schnurr and Dondero (1993, Intervirology; 36:79-83); Jong et al. (1999, J Clin Microbiol., 37:3940-3945). The human serotypes have been categorized into six subgenera (A-F) based on a number of biological, chemical, immunological and structural criteria which include hemagglutination properties of rat and rhesus monkey erythrocytes, DNA homology, restriction enzyme cleavage patterns, percentage G+C content and oncogenicity; Straus, supra; Horwitz, supra. A given serotype can be identified by a number of methods including restriction mapping of viral DNA; analyzing the mobility of viral DNA; analyzing the mobility of virion polypeptides on SDS-polyacrylamide gels following electrophoresis; comparison of sequence information to known sequence particularly from capsid genes (e.g., hexon) which contain sequences that define a serotype; and comparing a sequence with reference sera for a particular serotype available from the ATCC. Classification of adenovirus serotypes by SDS-PAGE has been discussed in Wadell et al. (1980, Ann. N.Y. Acad. Sci. 354:16-42). Classification of adenovirus serotypes by restriction mapping has been discussed in Wadell et al.(1984, Current Topics in Microbiology and Immunology 110:191-220).

Adenovirus has been a very attractive target for delivery of exogenous genes. The biology of adenoviruses is very well understood. Adenovirus has not been found to be associated with severe human pathology in immuno-competent individuals. The virus is extremely efficient in introducing its DNA into the host cell and is able to infect a wide variety of cells. The virus can be produced at high virus titers in large quantities. The adenovirus genome is very well characterized. It consists of a linear double-stranded DNA molecule of approximately 36,000 base pairs, and despite the existence of several distinct serotypes, there is some general conservation in the overall organization of the adenoviral genome with specific functions being similarly positioned. Furthermore, the virus can be rendered replication defective by deletion of the essential early-region 1 (E1) of the viral genome (Brody et al, 1994, Ann NY Acad Sci., 716:90-101). Replication-defective adenovirus vectors have been used extensively as gene transfer vectors for vaccine and gene therapy purposes. These vectors are propagated in cell lines that provide E1 gene products in trans. Supplementation of the essential E1 gene products in trans is very effective when the vectors are from the same or a very similar serotype. E1-deleted group C serotypes (Ad1, Ad2, Ad5 and Ad6), for instance, grow well in 293 or PER.C6 cells which contain and express the Ad5 E1 region. Presently, two well-characterized adenovirus serotypes from subgroup C, Ad5 and Ad2, are the most widely used gene delivery vectors. However, the Ad5 E1 sequences in 293 or PER.C6 cells do not fully complement the replication of all serotypes other than group C. An efficient means for the propagation and rescue of alternative serotypes in an Ad5 E1-expressing cell line (such as PER.C6 or 293) was disclosed in pending U.S. provisional application (Ser. No. 60/405,182, filed Aug. 22, 2002). This method involves the incorporation of a critical E4 region into the adenovirus to be propagated. The critical E4 region is native to a virus of the same or highly similar serotype as that of the E1 gene product(s), particularly the E1B 55K region, of the complementing cell line, and comprises, in the least, nucleic acid encoding E4 Orf6.

The present invention relates in part to a preserved formulation comprising a live adenovirus or live recombinant adenovirus particle (such as a replication-deficient adenovirus particle carrying a transgene expressing an HIV antigen) which further comprises a preservative to allow for a multi-dose image. To this end, the use of the term “adenovirus” is meant to cover any virus that is substantially a live adenovirus, including but not limited to known mammalian serotypes of adenovirus (such as human serotypes discussed herein as well as other such mammalian serotypes, such as those found in non-human primates), as well as recombinant forms of such mammalian forms and serotypes of adenovirus which are utilized in vaccine and gene therapy applications (e.g., known replication-deficient adenovirus vectors which comprise a transgene which upon host administration, expresses an antigen of interest to generate either an immune response against that antigen or to treat an existing disease or disorder). So again, a “live adenovirus” or “live adenovirus vaccine” or the like is meant to include, but not necessarily be limited to, virulent serotypes of known adenoviruses (e.g., wild type or modified forms of various adenovirus serotypes as discussed infra), live attenuated adenovirus-based vaccines (e.g., viral vaccines which are live but non-pathogenic due to reduced virulence, usually by serial passage of the pathogen through cell culture techniques) and a live recombinant adenovirus vaccine which will contain a gene(s) or portions thereof which encode for a immunogenic protein or peptide which is expressed upon in vivo administration of this recombinant vectored vaccine). Again, an exemplified form of the latter can be found throughout the Example sections, namely MRKAd5gag. Of course, the invention is in no way limited to such an exemplified adenoviral vaccine vector and/or particular formulation. Instead, as noted above, the artisan will be able to use the teaching of this specification to adequately preserve a live virus, especially a live adenovirus or live adenovirus-based recombinant vaccine, regardless of the specific antigen(s) expressed in vivo. Therefore, any formulated live mammalian adenovirus or live recombinant mammalian adenovirus vector (either as a vaccine or gene therapy candidate) which provides adequate viral stability (for at least approximately 1-2 years at 2-8° C.) is a candidate for preservation, and hence, multi-dose filling. The artisan will be able to utilize the teachings herein to choose a formulation that provides the best balance between viral stability and viral preservation, thus allowing for a multi-dose filing strategy. As noted above, a preferable formulation candidate would be one that affords a level of adenovirus stability for at least approximately 1-2 years at 2-8° C. Examples of previously described adenovirus formulation include, but are not meant to be limited to a virus formulation which (1) contains glycerol, sodium phosphate, Tris, sucrose, MgCl₂, and polysorbate 80 (see WO 99/41416); (2) a virus formulation with concentrations of sucrose from about 0.75M to 1.5M sucrose (see WO98/02522); (3) frozen liquid adenoviral formulations containing Tris, sucrose and MgCl₂ (see Nyberg-Hoffman et al., 1999, Nature Medicine 5 (8): 955-956); and/or (4) a lyophilized, frozen liquid and liquid virus formulations that contain Tris and phosphate buffered solutions with high concentrations of sucrose, trehalose or sorbitol/gelatin (see Croyle et al. (1998, Pharm. Dev. Technol. 3 (3): 373-383).

As noted supra, preferred population of formulation candidates to preserve an adenovirus are liquid adenovirus formulations which show improved stability when stored in about the 2-8° C. range while also being compatible with parenteral administration. These formulations may comprise a buffer, a sugar as a cryoprotectant, a salt, a divalent cation, a non-ionic detergent, as well as at least one free radical scavenger and/or chelating agent to inhibit free radical oxidation. The family of stabilizing virus formulations are disclosed within PCT International Application PCT/US01/07194 (International Publication No. WO 01/66137), hereby incorporated by reference in its entirety. Briefly, these formulations have shown to provide stability to adenovirus at varying degrees of virus concentration and may be administered to a variety of vertebrate organisms, preferably mammals and especially humans, as a recombinant adenovirus vaccine. Expected viral concentration in a single dose will preferably be in the range from about 1×10⁷ vp/mL (virus particles/milliliter) to about 1×10¹³ vp/mL. A more preferred range is from about 1×10⁹ to 1×10¹² vp/mL, with an especially preferred virus concentration being from about 1×10¹⁰ to 1×10¹² vp/mL. The effective amount for human administration may, of course, vary according to a variety of factors such as the individual's condition, weight, sex and age. Other factors include the mode of administration. The amount of expressible DNA to be administered to a human recipient will depend on the strength of the transcriptional and translational promoters used in the recombinant viral construct, and, if used as a vaccine, on the immunogenicity of the expressed gene product, as well as the level of pre-existing immunity to a virus such as adenovirus. Any such formulation is a candidate for addition of a preservative so as to generate a multi-dose image for the vaccine. Such a live virus formulation will contain a physiologically acceptable buffer, preferably but not necessarily limited to a formulation buffered with Tris (trimethamine), histidine, phosphate, citrate, succinate, acetate, glycine, and borate, within a pH range including but not limited to about 6.0 to about 9.0, preferably a pH range from about 6.4 to about 7.4.

A centerpiece of the formulations from WO 01/66137 was the inclusion of components that act as inhibitors of free radical oxidation. Such formulations, as exemplified but in no way limited by the discussion herein, as well as the listing in Table 1, comprise components which may inhibit free radical oxidation further enhance the stability characteristics of the core adenoviral formulations disclosed herein. Free radical oxidation inhibitors which may be utilized include but are not necessarily limited to ethanol (EtOH), EDTA, an EDTA/ethanol combination, triethanolamine (TEOA), mannitol, histidine, glycerol, sodium citrate, inositol hexaphosphate, tripolyphosphate, succinic and malic acid, desferal, ethylenediamine-Di(o-hydoxy-phenylacetic acid (EDDHA) and diethylenetriaminepenta-acetic acid (DTPA), or specific combinations thereof. It is preferred that the inhibitor of free radical oxidation be either an EDTA/EtOH combination, EtOH alone, and/or histidine, and combinations of these compounds thereof. It is shown herein that the combination with other components may determine the effectiveness of the free radical oxidation inhibitor. For example, the combination of EDTA/EtOH is shown to be very effective at increasing stability, while DTPA (alone) in the absence of MgCl₂ also enhances stability. As noted in WO 01/66137, the skilled artisan may “mix and match” various components, in some cases a scavenger and a chelator are required, while in other formulations only a chelator may be required. Preferably, the choice of chelator will determine whether or not the addition of a scavenger is needed. Additional free radical scavengers and chelators are known in the art and apply to the preserved formulations and methods of use described herein. An essential quality of these formulations is that non-reducing free radical scavengers and/or chelators are important for maximizing both short and long term stability of viral formulations, especially recombinant adenoviral formulations. These formulations have been shown to be stable for extended periods of time (2 years or more) at temperatures up through the 2-8° C. range, or higher, when compared to core formulations which do not contains these inhibitors. In addition, these formulations are compatible with parenteral administration. These characteristics make this series of viral formulations one preferred choice as candidates for use in a multi-dose vaccination regime, and hence, candidates for addition of a preservative as described herein.

Components and concentration ranges for these core candidate formulations for preservation and a multi-dose administration regime include but are not limited to the following:

(1) buffer, pH—about 1 mM to about 20 mM Tris, (trimethamine), histidine (which also acts as an oxidation inhibitor), phosphate, citrate, succinate, acetate, glycine, and borate, or a combination (e.g., such as 10 mM Tris and 10 mM histidine in A195) within a pH range including but not limited to about 6.0 to about 9.0.

(2) cryoprotectant, salt, osmolarity

:cryoprotectants—include but are not limited to polyhydroxy hydrocarbons such as sorbitol, mannitol, glycerol and dulcitol and/or disaccharides such as sucrose, lactose, maltose or trehalose.

:salts—including but not necessarily limited to sodium chloride, potassium chloride, sodium sulfate, and potassium sulfate, present at an ionic strength which is physiologically acceptable to the host. A purpose of inclusion of a salt in the formulation is to attain the desired ionic strength or osmolarity. Contributions to ionic strength may come from ions produced by the buffering compound as well as from the ions of non-buffering salts. A preferred salt, NaCl, is present from a range rising up to about 250 mM, the sucrose and NaCl concentrations being complementary such that the total osmolarity ranges from about 200 mOs/L to about 800 mOs/L, as noted infra;

:osmolarity—a useful range of total osmolarity which both promotes long term stability at temperature of 2-8° C., or higher, while also making the formulation useful for parenteral, and especially intramuscular, injection. To this end the effective range of total osmolarity (the total number of molecules in solution) is from about 200 mOs/L to about 800 mOs/L, with a preferred range from about 250 mOs/L to about 450 mOs/L. An especially preferred osmolarity for the formulations disclosed herein is about 300 mOs/L. A salt free formulation may contain from about 5% to about 25% sucrose, with a preferred range of sucrose from about 7% to about 15%, with an especially preferred sucrose concentration in a salt free formulation being from 10% to 12%. Alternatively, a salt free sorbitol-based formulation may contain sorbitol within a range from about 3% to about 12%, with a preferred range from about 4% to 7%, and an especially preferred range is from about 5% to about 6% sorbitol in a salt-free formulation. Salt-free formulations will of course warrant increased ranges of the respective cryoprotectant in order to maintain effective osmolarity levels. To again utilize sucrose and sorbitol as examples, and not as a limitation, an effective range of a sucrose-based solution in 75 mM NaCl is from about 2% about 8% sucrose, while a sorbitol-based solution in 75 mM NaCl is from about 1% to about 4% sorbitol.

(3) divalent cation—including but not limited to MgCl₂, CaCl₂ and MnCl₂ at a concentration ranging from about 0.1 mM to about 10 mM, with up to about 5 mM being preferred.

(4) non-ionic surfactant—a non-ionic surfactant for use in the preserved formulations of the present invention include but are not limited to polyoxyethylene sorbitan fatty acid esters, including but not limited to Polysorbate-80 (Tween 80®), Polysorbate-60 (Tween 60®), Polysorbate-40 (Tween 40®) and Polysorbate-20 (Tween 20®), polyoxyethylene alkyl ethers, including but not limited to Brij 58®, Brij 35®, as well as others such as Triton X-100®, Triton X-114®, NP40®, Span 85 and the Pluronic series of non-ionic surfactants (e.g., Pluronic 121), with preferred components Polysorbate-80 at a concentration from about 0.001% to about 2% (with up to about 0.25% being preferred) or Polysorbate-40 at a concentration from about 0.001% to 1% (with up to about 0.5% being preferred).

(5) free radical scavenger/chelating agent—Inhibitors of free radical oxidation include but are not limited to ethanol (EtOH), EDTA, an EDTA/ethanol combination, triethanolamine (TEOA), mannitol, histidine, glycerol, sodium citrate, inositol hexaphosphate, tripolyphosphate, succinic and malic acid, desferal, ethylenediamine-Di(o-hydoxy-phenylacetic acid (EDDHA) and diethylenetriaminepenta-acetic acid (DTPA) are contemplated. In the above-described formulations, at least one non-reducing free radical scavenger may be added to concentrations which effectively enhance stability of the core formulation. Especially useful ranges include (i) EDTA from about 1 μM to about 500 μM, preferably in a range from about 50 μM to about 250 μM, and an especially preferred concentration of at or around 100 μM; (ii) ethanol from about 0.1% to about 5.0%, preferably in a range from about 0.25% to about 2.0%, and an especially preferred amount totaling at or around 0.5%; (iii) DTPA from about 1 μM to about 500 μM, preferably in a range from about 50 μM to about 250 μM, and an especially preferred concentration at or around 100 μM; (iv) CaCl₂ from about 0.1 mM to about 10 mM, preferably in a range from about 0.5 mM to about 5 mM, and an especially preferred concentration at or around 1 mM; (v) sodium citrate from about 1 mM to about 100 mM, preferably in a range from about 5 mM to about 25 mM, and an especially preferred concentration at or around 10 mM; and, (vi) histidine at about 1 mM to about 20 mM; or combinations thereof. These inhibitors of free radical oxidation may also be added in various combinations, including but not limited to two scavengers, a sole, or possible a sole scavenger in the absence of another component, such as a divalent cation. The skilled artisan may “mix and match” various components, in some cases a scavenger and a chelator are required, while other formulations only a chelator may be required. Preferably, the choice of chelator will determine whether or not the addition of a scavenger is needed. Additional free radical scavengers and chelators are known in the art and apply to the formulations and methods of use described herein.

To this end, specific embodiments of the stable and preserved adenovirus-based formulations of the present invention are formulations which cover ranges and/or combination which can be contemplated by review of Table 1, as shown below. Each of the formulations contemplated in Table 1 and elsewhere in the specification becomes a candidate preservation formulation for live adenovirus by addition of chlorobutanol at a range up to the maximum (i.e., highest [CB]) effective solubility of chlorobutanol for the respective live viral formulation.

TABLE 1

| Form. # | Description |
|---|---|
| A101 | 10 mM Tris, 10% glycerol (v/v), 1 mM MgCl₂, pH 7.5 |
| A102 | 6 mM phosphate, 150 mM NaCl, 10% glycerol (v/v), pH 7.2 |
| A103 | 6 mM phosphate, 150 mM NaCl, pH 7.2 |
| A104 | 5 mM Tris, 150 mM NaCl, 1 mM MgCl₂, 0.005% PS-80, pH 8.0 |
| A105 | 5 mM Tris, 75 mM NaCl, 5% sucrose (w/v), 1 mM MgCl₂, 0.005% PS-80, pH 8.0 |
| A106 | 5 mM Tris, 14% sucrose (w/v), 1 mM MgCl₂, 0.005% PS-80, pH 8.0 |
| A107 | 5 mM Tris, 8% sorbitol (w/v), 1 mM MgCl₂, 0.005% PS-80, pH 8.0 |
| A108 | 5 mM Tris, 75 mM NaCl, 5% sucrose (w/v), 0.005% PS-80, pH 8.0 |
| A109 | 5 mM Tris, 75 mM NaCl, 5% sucrose (w/v), 1 mM MgCl₂, pH 8.0 |
| A110 | 5 mM Tris, 75 mM NaCl, 5% sucrose (w/v), 1 mM MgCl₂, 0.02% PS-80, pH 8.0 |
| A111 | 5 mM Tris, 75 mM NaCl, 5% sucrose (w/v), 1 mM MgCl₂, 0.1% PS-80, pH 8.0 |
| A112 | 5 mM Tris, 75 mM NaCl, 5% sucrose (w/v), 1 mM MgCl₂, 0.005% PS-80, |
| | 100 μm DTPA, pH 8.0. |
| A113 | 5 mM Tris, 75 mM NaCl, 5% sucrose (w/v), 1 mM MgCl₂, 0.005% PS-80, 100 μM EDTA, 0.5% |
| | EtOH, pH 8.0 |
| A114 | 5 mM Tris, 75 mM NaCl, 5% sucrose (w/v), 1 mM MgCl₂, 0.005% PS-80, 1.0 mM TEOA, pH 8.0 |
| A115 | 5 mM Tris, 75 mM NaCl, 5% sucrose (w/v), 1 mM MgCl₂, 0.005% PS-80, 10 mM sodium citrate, |
| | pH 8.0 |
| A116 | 5 mM Tris, 75 mM NaCl, 5% sucrose (w/v), 0.005% PS-80, 100 μM DTPA, pH 8.0 |
| A117 | 5 mM Tris, 75 mM NaCl, 5% sucrose (w/v), 0.005% PS-80, 100 μM EDTA, |
| | 0.5% EtOH, pH 8.0 |
| A118 | 5 mM Tris, 75 mM NaCl, 5% sucrose (w/v), 0.005% PS-80, 1.0 mM TEOA, pH 8.0 |
| A119 | 5 mM Tris, 75 mM NaCl, 5% sucrose (w/v), 0.005% PS-80, 10 mM sodium citrate, pH 8.O |
| A120 | 5 mM Tris, 75 mM NaCl, 5% sucrose (w/v), 0.005% PS-80, 100 μM EDTA, 0.5% EtOH, 1 mM |
| | CaCl₂, pH 8.0 |
| A121 | 5 mM Tris, 5% sucrose (w/v), 1 mM MgCl₂, 3% (w/v) mannitol, 0.005% PS-80, |
| | pH 8.0 |
| A125 | 5 mM Tris, 75 mM NaCl, 5% sucrose (w/v), 1 mM MgCl₂, 10 mM ascorbic acid, 0.005% PS-80, |
| | pH 8.0 |
| A126 | 5 mM Tris, 75 mM NaCl, 5% sucrose (w/v), 1 mM MgCl₂, 0.05% PS-80, pH 8.0 |
| A127 | 5 mM Tris, 75 mM NaCl, 5% sucrose (w/v), 1 mM MgCl₂, 0.15% PS-80, pH 8.0 |
| A128 | 5 mM Tris, 75 mM NaCl, 5% sucrose (w/v), 1 mM MgCl₂, 0.005% PS-40, pH 8.0 |
| A129 | 5 mM Tris, 75 mM NaCl, 5% sucrose (w/v), 1 mM MgCl₂, 0.1% PS-40, pH 8.0 |
| A130 | 5 mM Tris, 75 mM NaCl, 5% sucrose (w/v), 2 mM MgCl₂, 0.005% PS-80, pH 8.0 |
| A131 | 5 mM Tris, 75 mM NaCl, 5% sucrose (w/v), 5 mM MgCl₂, 0.005% PS-80, pH 8.0 |
| A132 | 5 mM Tris, 75 mM NaCl, 5% sucrose (w/v), 1 mM MgCl₂, 0.005% PS-80, |
| | 0.5% EtOH, pH 8.0 |
| A133 | 5 mM Tris, 75 mM NaCl, 5% sucrose (w/v), 1 mM MgCl₂, 0.005% PS-80, |
| | 100 μM EDTA, pH 8.0 |
| A134 | 5 mM Tris, 75 mM NaCl, 5% sucrose (w/v), 1 mM MgCl₂, 0.005% PS-80, |
| | 1.0% EtOH, pH 8.0 |
| A135 | 5 mM Tris, 75 mM NaCl, 5% sucrose (w/v), 1 mM MgCl₂, 0.005% PS-80, |
| | 100 μM EDTA, 1.0% EtOH, pH 8.0 |
| A136 | 5 mM Tris, 75 mM NaCl, 5% sucrose (w/v), 1 mM MgCl₂, 0.1% PS-80, |
| | 100 μM EDTA, 0.5% EtOH, pH 8.0 |
| A137 | 5 mM Tris, 75 mM NaCl, 5% sucrose (w/v), 1 mM MgCl₂, 0.005% PS-80, |
| | 1 mg/ml plasmid DNA comprising an HIV-1 gag sequence, pH 8.0A138 |
| A138 | 5 mM Tris, 75 mM NaCl, 5% sucrose (w/v), 1 mM MgCl₂, 0.005% PS-80, 100 μM EDTA, 0.5% |
| | EtOH, 1 mg/ml plasmid DNA comprising an HIV-1 gag sequence, pH 8.0 |
| A149 | 5 mM Tris, 75 mM NaCl, 2.7% (w/v) mannitol, 1 mM MgCl₂, 0.005% PS-80, 100 μM EDTA, |
| | 0.5% EtOH, pH 8.0 |
| A151a | 5 mM Tris, 75 mM NaCl, 5% sucrose (w/v), 1 mM MgCl₂, 0.005% PS-80, 100 μM EDTA, 0.5% |
| | EtOH, 5 mM histidine, pH 8.0 |
| A151b | 5 mM Tris, 75 mM NaCl, 5% sucrose (w/v), 1 mM MgCl₂, 0.005% PS-80, 100 μM EDTA, 0.5% |
| | EtOH, 5 mM histidine, pH 7.5 at 30° C. |
| A152 | 5 mM Tris, 75 mM NaCl, 5% sucrose (w/v), 2 mM MgCl₂, 0.1% PS-80, 100 μM EDTA, 0.5% |
| | EtOH, 5 mM histidine, 5 mM TEOA, pH 7.5 at 30° C. |
| A153 | 5 mM Tris, 75 mM NaCl, 5% sucrose (w/v), 2 mM MgCl₂, 0.1% PS-80, 100 μM EDTA, 0.5% |
| | EtOH, 5 mM histidine, 5 mM TEOA, 5% (v/v) glycerol, pH 7.5 at 30° C. |
| A155 | 15 mM Tris, 75 mM NaCl, 5% sucrose (w/v), 1 mM MgCl₂, 0.005% PS-80, 100 μM EDTA, 0.5% |
| | EtOH, pH 8.0 |
| A159 | 5 mM Tris, 75 mM NaCl, 2.7% mannitol (w/v), 1 mM MgCl₂, 0.005% PS-80, 100 μM EDTA, |
| | 0.5% EtOH, 5 mM histidine, pH 8.0 |
| A160 | 5 mM Tris, 75 mM NaCl, 2.7% mannitol (w/v), 1 mM MgCl₂, 0.005% PS-80, 100 μM EDTA, 5 |
| | mM histidine, pH 8.0 |
| A165 | 5 mM Tris, 75 mM NaCl, 5% sucrose (w/v), 2 mM MgCl₂, 0.1% PS-80, 100 μM EDTA, 0.5% |
| | EtOH, 5 mM histidine, pH 7.5 at 30° C. |
| A166 | 10 mM Tris, 75 mM NaCl, 5% sucrose (w/v), 1 mM MgCl₂, 0.1% PS-80, 100 μM EDTA, 0.5% |
| | EtOH, 7.5 mM histidine, 1 mM TEOA, pH 7.6 |
| A167 | 10 mM Tris, 75 mM NaCl, 5% sucrose (w/v), 1 mM MgCl₂, 0.1% PS-80, 100 μM EDTA, 0.5% |
| | EtOH, 10 mM histidine, 1 mM TEOA, pH 8.0 |
| A168 | 10 mM Tris, 75 mM NaCl, 5% sucrose (w/v), 1 mM MgCl₂, 0.1% PS-80, 100 μM EDTA, 0.5% |
| | EtOH, 7.5 mM histidine, 1 mM TEOA, 1.0% mannitol, pH 7.7 |
| A169 | 10 mM Tris, 75 mM NaCl, 5% sucrose (w/v), 1 mM MgCl₂, 0.1% PS-80, 100 μM EDTA, 0.5% |
| | EtOH, 10 mM histidine, 1 mM TEOA, 1% mannitol, pH 8.0 |
| A170 | 10 mM Tris, 75 mM NaCl, 5% sucrose (w/v), 1 mM MgCl₂, 0.1% PS-80, 100 μM EDTA, |
| | 0.5% EtOH, 10 mM histidine, pH 8.0 |
| A171 | 10 mM Tris, 75 mM NaCl, 5% sucrose (w/v), 0.1% PS-80, 100 μM EDTA, 0.5% EtOH, |
| | 10 mM histidine, 1 mM TEOA, 1% mannitol, pH 8.0 |
| A172 | 10 mM Tris, 75 mM NaCl, 5% sucrose (w/v), 0.005% PS-80, 100 μM EDTA, 0.5% EtOH, pH 8.0 |
| A173 | 10 mM Tris, 75 mM NaCl, 5% sucrose (w/v), 0.005% PS-80, 100 μM EDTA, 0.5% EtOH, |
| | 10 mM histidine, pH 8.0 |
| A174 | 10 mM Tris, 75 mM NaCl, 5% sucrose (w/v), 0.1% PS-80, 100 μM EDTA, 0.5% EtOH, |
| | 7.5 mM histidine, 1 mM TEOA, pH 7.62 |
| A175 | 10 mM Tris, 75 mM NaCl, 5% sucrose (w/v), 1 mM MgCl2, 0.1% PS-80, 100 μM EDTA, |
| | 0.5% EtOH, 7.5 mM histidine, pH 7.62 |
| A176 | 10 mM Tris, 75 mM NaCl, 5% sucrose (w/v), 0.1% PS-80, 100 μM EDTA, 0.5% EtOH, |
| | 7.5 mM histidine, pH 7.62 |
| A178 | 10 mM Tris, 75 mM NaCl, 5% sucrose (w/v), 1 mM MgCl2, 0.005% PS-80, 100 μM EDTA, |
| | 0.5% EtOH, 7.5 mM histidine, 1 mM TEOA, pH 7.62 |
| A179 | 10 mM Tris, 75 mM NaCl, 5% sucrose (w/v), 1 mM MgCl2, 0.01% PS-80, 100 μM EDTA, |
| | 0.5% EtOH, 7.5 mM histidine, 1 mM TEOA, pH 7.62 |
| A180 | 10 mM Tris, 75 mM NaCl, 5% sucrose (w/v), 1 mM MgCl2, 0.02% PS-80, 100 μM EDTA, |
| | 0.5% EtOH, 7.5 mM histidine, 1 mM TEOA, pH 7.62 |
| A181 | 10 mM Tris, 75 mM NaCl, 5% sucrose (w/v), 1 mM MgCl2, 0.05% PS-80, 100 μM EDTA, |
| | 0.5% EtOH, 7.5 mM histidine, 1 mM TEOA, pH 7.62 |
| A182 | 5 mM Tris, 75 mM NaCl, 5% sucrose (w/v), 1 mM MgCl2, 0.005% PS-80, 7.5 mM |
| | histidine, pH 8.0 |
| A183 | 5 mM Tris, 75 mM NaCl, 5% trehalose (w/v), 1 mM MgCl2, 0.005% PS-80, pH 8.0 |
| A184 | 10 mM Tris, 75 mM NaCl, 5% sucrose (w/v), 1 mM MgCl2, 0.1% PS-80, 100 μM EDTA, |
| | 0.5% EtOH, 10 mM histidine, pH 7.62 |
| A184b | 10 mM Tris, 75 mM NaCl, 5% sucrose (w/v), 1 mM MgCl2, 0.1% PS-80, 100 μM EDTA, |
| | 0.5% EtOH, 10 mM histidine, pH 7.4 |
| A195 | 10 mM Tris, 75 mM NaCl, 5% sucrose (w/v), 1 mM MgCl2, 0.02% PS-80, 100 μM EDTA, |
| | 0.5% EtOH, 10 mM histidine, pH 7.4 |

As noted supra, one exemplified virus component which remains stable and is in fact preserved is an Ad5 replication deficient virus which contains an open reading frame for the HIV p55 gag antigen. It is evident that the present invention is not limited to any single formulation comprising such an adenovirus vaccine vector. Instead, as noted above, any mammalian adenovirus or adenovirus-like entity is a candidate for preservation. The artisan need only choose an appropriate adenovirus or adenovirus vector, a formulation which provides adequate stability for the respective adenovirus or adenovirus vector, and then proceed to test preservation ability, either with assays shown herein or by other methodology which will determine the ability of a specific compound to act to preserve a specific adenoviral formulation. While the preserved formulations of the present invention are exemplified by recombinant adenovirus carrying HIV genes encoding gag, pol and nef, respectively, the invention is in no way, shape or form limited to such formulations. On one level, any additional HIV gene (e.g., such as env, rev, tat, vpr, vpu, vif and/or pro) may be formulated as disclosed herein. Examples of such adenoviral constructs are disclosed in PCT International Applications PCT/US00/18332 (WO 01/02607) and PCT International Applications PCT/US01/28861 (WO 02/22080), both of which are hereby incorporated by reference. Such formulations may be of a single dose image with a single recombinant adenoviral vector, or may be possess a single dose image but be formulated with more than one distinct live recombinant viral vector (e.g., MRKAd5gag, MRKAdpol and/or MRKAd5nef). Alternatively, such formulations may be of a multi-dose image with a single recombinant adenoviral vector, or may be possess a multi-dose image but be formulated with more than one distinct live recombinant viral vector (e.g., MRKAd5gag, MRKAdpol and/or MRKAd5nef). Of course, any non-HIV gene for vaccine or gene therapy applications will also fall within the scope the these teachings, and in turn will be candidates for being filled in single or multi-dose vials as part of a live recombinant virus, and especially a live recombinant adenovirus vector in a chlorobutanol-containing formulation.

The short-term stability studies of the adenovirus-based HIV vaccine containing FDA approved preservatives for injectables show that chlorobutanol and benzoic acid is compatible with adenovirus. An Arrhenius plot based on these short-term data indicates that the projected loss of adenovirus infectivity in A195 is ≦0.1 logs after 2-year of 2-8° C. storage, with or without the addition of chlorobutanol (0.5% w/v).

The HIV vaccine immunogenicity testing in mice (see Example 3) further confirms that chlorobutanol at 0.4% has no effect on the vaccine potency ill vivo. Based on accelerated stability, antimicrobial effectiveness (AME) and immunogenicity testing, the development of a multi-dose adenovirus-based HIV vaccine containing chlorobutanol is feasible. These examples exemplify, but in no way limit, a formulation of the present invention, which is a formulation described in Table 2, or any relevant component combinations thereof, which contain chlorobutanol at a concentration up to about 0.6%. Another preferred formulation would be an A195-based formulation to the extent of comprising a biologically acceptable concentration of a buffer (tris and histidine [histidine also being a free radical scavenger]), a sugar (sucrose), a salt (NaCl), a divalent cation (MgCl₂), a surfactant (PS-80), a chelator (EDTA), a free radical scavenger (ethanol), and chlorobutanol at an acceptable concentration, such as up to about 0.6% (see Table 3). An exemplified adenovirus formulation of the present invention include, but is in no way is limited to A502, a formulation comprising 10 mM Tris, 10 mM histidine, pH 6.8 at 20-23° C., 5% (w/v) sucrose, 75 mM NaCl, 1 mM MgCl₂, 0.02% (v/v) PS-80, 0.1 mM EDTA, 0.5% (v/v) ethanol, and 0.5% chlorobutanol.

The following examples are provided to illustrate the present invention without, however, limiting the same hereto.

### EXAMPLE 1

### Stability of Adenovirus in the Presence of Preservative

The live adenovirus vector and formulation used to exemplify the present invention is as follows: a MRKAd5gag, MRKAdpol and MRKAd5nef construct (as disclosed in WO 02/22080) is a non-infectious group C adenovirus serotype 5 (Ad5) vector with a transgene encoding HIV proteins. The vaccine is a clear solution formulated in A195 buffer for the refrigerated storage and intramuscular administration (Table 2). Ad5 in A195 is stable for at least 18 months at 2-8° C. and is projected to lose ≦0.1 logs of infectivity after 2 years of 2-8° C. storage.

TABLE 2
A195 Formulation Description

| Excipient | Function of Excipient |
|---|---|
| 10 mM Tris | Buffer |
| 10 mM Histidine | Buffer, Oxidation Inhibitor |
| pH 7.4 at 25° C. | pH Optimized for Stability |
| 5% (w/v) sucrose | Cryoprotectant |
| 75 mM NaCl | Osmolarity Adjustment |
| 1 mM MgCl₂ | Stability |
| 0.02% (v/v) PS-80 | Stability/Prevent Adsorption |
| 0.1 mM EDTA | Stability (metal ion chelator) |
| 0.5% (v/v) Ethanol | Stability (free radical scavenger) |

Table 3 shows exemplified preserved live adenovirus formulations as tested within these Example section.

TABLE 3
Exemplified Preserved Live Adenovirus Formulations

| | Chlorobutanol | pH | Tris | Histidine | Sucrose | PS-80 | NaCl | MgCl₂ | EDTA | Ethanol |
|---|---|---|---|---|---|---|---|---|---|---|
| A501 | 0.4% (w/v) CB | 6.80 | 10 mM | 10 mM | 5% (w/v) | 0.02% (v/v) | 75 mM | 1 mM | 0.1 mM | 0.5% (v/v) |
| A501a | 0.4% (w/v) CB | 7.40 | 10 mM | 10 mM | 5% (w/v) | 0.02% (v/v) | 75 mM | 1 mM | 0.1 mM | 0.5% (v/v) |
| A501b | 0.4% (w/v) CB | 6.40 | 10 mM | 10 mM | 5% (w/v) | 0.02% (v/v) | 75 mM | 1 mM | 0.1 mM | 0.5% (v/v) |
| A501c | 0.4% (w/v) CB | 6.00 | 10 mM | 10 mM | 5% (w/v) | 0.02% (v/v) | 75 mM | 1 mM | 0.1 mM | 0.5% (v/v) |
| A502 | 0.5% (w/v) CB | 6.80 | 10 mM | 10 mM | 5% (w/v) | 0.02% (v/v) | 75 mM | 1 mM | 0.1 mM | 0.5% (v/v) |
| A502a | 0.5% (w/v) CB | 7.40 | 10 mM | 10 mM | 5% (w/v) | 0.02% (v/v) | 75 mM | 1 mM | 0.1 mM | 0.5% (v/v) |
| A502b | 0.5% (w/v) CB | 6.40 | 10 mM | 10 mM | 5% (w/v) | 0.02% (v/v) | 75 mM | 1 mM | 0.1 mM | 0.5% (v/v) |
| A502c | 0.5% (w/v) CB | 6.00 | 10 mM | 10 mM | 5% (w/v) | 0.02% (v/v) | 75 mM | 1 mM | 0.1 mM | 0.5% (v/v) |

Materials and Methods—Adenovirus Lots:
1. MRKAd5gag0202ASFP, stock 1.12×10¹² VP/ml;
2. MRKAd5nef0102ASFP, stock 1.02×10¹² VP/ml;
3. MRKAd5gag0010SFFP, stock 1.67×10¹² VP/ml;
4. MRKAd5pol0221A, stock 8.39×10¹¹ VP/ml.

QPA Assay for Adenovirus Infectivity—The compatibility of preservatives with adenovirus was assessed by measuring the infectivity adenovirus using QPA assay. The QPA assay is based on the finding that the quantity of replicating adenoviral genomes, upon infection of 293 cells, is proportional to the input quantity of infectious adenovirus 24 hours post-infection (P.I.). The accumulated adenoviral genomes are purified using Qiagen 96-well Blood DNA Extraction Kit 24 hrs P.I. and quantitated using Perkin-Elmer TaqMan PCR technology. A standard curve is constructed by using the TaqMan PCR cycle threshold (C_{T}) reflecting the quantity of accumulated adenoviral genomes due to infection and subsequent replication as a function of the input viral infectivity of standard curve material that is determined by independent TCID₅₀ potency assay. The infectivity of samples is interpolated from the standard curve. Infectivity can be determined within 48-72 hours using this procedure. To determine the loss of adenovirus infectivity in stability samples the infectivity of the stability sample and the corresponding −70° C. control of the same formulation are determined during the same QPA run and on the same PCR plate. The loss of infectivity in the stability samples is expressed as log loss of infectivity compared to the −70° C. control.

Antimicrobial Effectiveness Testing—This set of experiments will demonstrate that the addition of a suitable preservative or preservatives provides adequate protection from adverse effects that may arise from microbial contamination or proliferation during storage and use of the preparation. The efficacy of the antimicrobial activity is demonstrated using the antimicrobial effectiveness (AME) testing, as described in US Pharmacopeia <51>, European Pharmacopoeia 5.1.3, and PAC Microbiology Method WGM_{—}066 (Rev.3). The criteria for passing test results are shown in Table 4, as follows:

TABLE 4
Antimicrobial Effectiveness (AME) Testing Criteria
Number of Log Reduction in Microbial
Population

| | Inoculum | | | | | |
|---|---|---|---|---|---|---|
| | (cfu) | 6 h | 24 h | 7 d | 14 d | 28 d |
| Bacteria^{a} | | | | | | |
| USP^{b} | 10⁵-10⁶ | — | — | 1 | 3 | NI^{c} |
| EP-A^{b} | 10⁶ | 2 | 3 | — | — | NR^{d} |
| EP-B^{b} | 10⁶ | — | 1 | 3 | — | NI |
| Fungi^{a} | | | | | | |
| USP | 10⁵-10⁶ | — | — | — | NI^{c} | NI^{c} |
| EP-A | 10⁶ | — | — | 2 | — | NI |
| EP-B | 10⁶ | — | — | — | 1 | NI |

^{a}The following were used as challenge organisms: Bacteria-S. aureus, P. aeruginosa, and E. coli; Yeast-C. albicans; Mold-A. niger.
^{b}JP acceptance criteria same as USP. EP-A is the recommended criteria. EP-B criteria replace A criteria in justified cases where A criteria cannot be attained.
^{c}NI = no increase, no more than 0.5 log unit higher than the previous value measured.
^{d}NR = no recovery.

HIV Vaccine Immunogenicity Test in Mice—MRKAd5gag in A195 containing the preservative was tested in mice to determine whether the preservative would have an impact on the immune response induced by MRKAd5gag, compared to MRKAd5gag in A195. Groups of BALB/c mice (5 to 10 mice per cohort) were injected intramuscularly with increasing doses of MRKAd5gag (10⁷, 10⁸, and 10⁹ VP) formulated in either A195 or A195 plus preservative. In each case, the vaccine was given as a 50 μL aliquot per quadriceps muscle; both muscles were treated. Serum samples were collected 3 weeks after the treatment and assayed for anti-HIV gag p24 titers using an established ELISA assay. At 6 weeks after dosing, spleens were collected from 5 mice per cohort, pooled and prepared for an IFN-gamma ELISPOT assay. In this case, T lymphocyte responses were induced against a known CD8⁺ gag epitope in BALB/c mice and against recombinant p24 antigen.

Results—Adenovirus was formulated in A195 buffer containing preservatives (i.e., the A500 series of Table 3). The infectivity of adenovirus was measured using a Q-PCR based Potency Assay (QPA) after storage at certain temperatures (2-8, 15, 20, 25, 30, and 37° C.) and compared to the control (corresponding sample stored at −70° C.). The stability of adenovirus was quantitatively presented as “Log Loss of Adenovirus Infectivity”. Variability of the QPA assay is reported to be +/−0.15 logs.

Benzyl Alcohol(BA)—Formulation buffer A195 containing 1% or 2% (v/v) benzyl alcohol was prepared and sterile filtered through 0.22 μm membrane. Adenovirus MRKAd5gag was added to each buffer at 10¹¹ VP/ml and stored as 1 ml/vial at 30° C. and −70° C. for 8 days. Samples were diluted 1000-fold using A105 buffer (see Table 1) for the QPA assay. Ad5 lost 0.26 logs of infectivity in A195 (control), but lost 1.11 logs in A195+0.1% BA and 1.63 logs in A195+0.2% BA. In addition, the infectivity of Ad5 in A195+0.2% BA at −70° C. was 1.6 logs lower than that of Ad5 in A195 buffer, probably due to the sample handling at room temperature. These data suggest that Ad5 has very poor stability in A195 buffer containing BA.

Phenol (P)—To investigate the effects of phenol on Ad5 stability, buffer A195 containing 0.44% (v/v) phenol was prepared. At room temperature, adenovirus MRKAd5nef was diluted into A195+0.44% P to a final concentration of 3×10⁸ VP/ml and stored in glass vials at −70, 2-8, 15, and 30° C., respectively. Samples were diluted 20-fold for the Adeno QPA assay. The following results suggest that Ad5 is very unstable in A195 buffer containing phenol: (1) the infectivity of Ad5 in A195+0.44% P (stored at −70° C.) was 1.9-2.7 logs lower than that of Ad5 in A195 buffer, (2) in A195 buffer, Ad5 lost only 0.26 logs of infectivity after 2-week storage at 30° C., (3) 0.05% (v/v) phenol in the sample tested had no significant effect on the cell-based QPA assay.

Benzethonium Chloride(BE) and Benzalkonium Chloride(BC)—The compatibility of adenovirus with BE and the other quaternary ammonium compound, benzalkonium chloride (BK), was tested. The concentration of preservative added to A195 buffer (pH 6.8) was 0.01% (w/v) BE and 0.02% (v/v) BK, respectively. Adenovirus MRKAd5pol was diluted into the buffers at a final concentration of 10¹⁰ VP/ml. The samples were stored at −70 and 37° C. for one week, then diluted 100-fold using A195 buffer (pH 6.8), and analyzed for infectivity using QPA assay. Compared to the −70° C. control, after storage at 37° C. for one week, the infectivity of Ad5 lost only 0.18 logs in A195, but lost 2.43 logs in A195+0.01% BE and 4.64 logs in A195+0.02% BK.

m-Cresol—(CR)-Preservative m-cresol (CR) has poor solubility in A195 buffer. The solution with 0.1% (v/v) CR is clear but 0.2% (v/v) CR in the solution is slightly turbid even after 5-hr of mixing at room temperature. The infectivity of Ad5 in A195+0.1% CR is 0.05-0.34 logs lower than Ad5 in A195 when stored at −70° C. No significant infectivity loss was observed when Ad5 was stored in A195+0.1% CR after 6-month at 15° C. However, the infectivity of Ad5 in Ad5+0.2% CR was >3 logs lower than that of Ad5 in A195 buffer.

Parabens—Methylparaben (MP) and propylparaben (PP): Parabens have poor solubility in aqueous solution: 2.5 g/L MP at 25° C. and 0.23 g/L PP at 15° C. A formulation buffer containing MP and PP at commonly used concentrations of 0.18% (w/v) and 0.02% (w/v), respectively, was prepared.

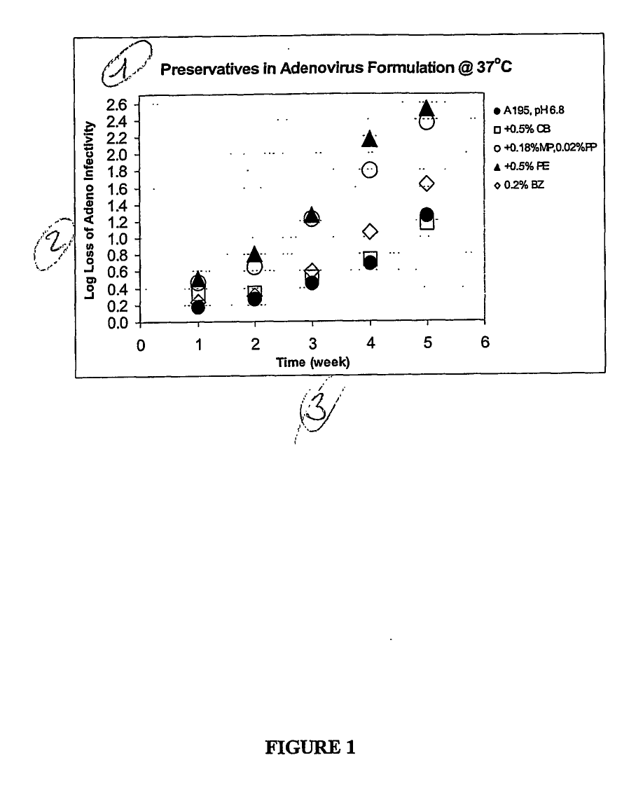

2-Phenoxyethanol (PE)—2-Phenoxyethanol has been used in killed vaccine products. The short-term stability data for Ad5 is shown in FIG. 1.

Chlorobutanol (CB) and Benzoic Acid (BZ)—Buffers of A195 (pH 6.8) containing 0.5% (w/v) chlorobutanol (CB) and 0.2% (w/v) benzoic acid (BZ), respectively, were prepared at room temperature, sterile filtered through 0.22 μm cellulose acetate membrane, and stored at 2-8° C. in the glass bottles. Adenovirus MRKAd5pol was diluted into the buffers at a final concentration of 10¹⁰ VP/ml. All samples were aliquoted into 3 ml glass vials at 1 ml/vial and stored at −70 and 37° C. respectively. FIG. 1 shows the log loss of adenovirus infectivity at 37° C. for 1-4 weeks vs. −70° C. storage. The infectivity of adenovirus was measured using QPA. From this short-term study under accelerated conditions, chlorobutanol and benzoic acid show no significant effects on the stability of adenovirus.

Summary—The stabilities of Ad5 in the presence of preservatives are categorized in Table 5. Both chlorobutanol and benzoic acid are compatible with Ad5 without any apparent damage to the live adenovirus assessed by infectivity assays conducted during short-term accelerated stability studies.

TABLE 5
Stability of adenovirus in the presence of preservatives

| Ad5 stability | Preservative | |
|---|---|---|
| No change | 0.5% | chlorobutanol |
| | 0.2% | benzoic acid |
| Significantly lower | 0.1% | m-cresol |
| | 0.18% | methylparaben |
| | 0.02% | propylparaben |
| | 0.5% | 2-phenoxyethanol |
| Very poor | 0.2% | m-cresol |
| | 1% | benzyl alcohol |
| | 0.44% | phenol |
| | 0.01% | benzethonium chloride |
| | 0.02% | benzalkonium chloride |

### EXAMPLE 2

### Antimicrobial Effectiveness Testing of A195+Preservative

The antimicrobial effectiveness (AME) should be demonstrated for the preservative containing product. An AME testing procedure is described in Table 4. For the AME testing of the multi-dose HIV vaccine formulation, only placebos were used in the testing, assuming adenovirus has no effect on the microbial growth during the testing. Replication deficient adenovirus (e.g., MRKAd5gag) is composed of protein and DNA. Total protein in a sample of 3×10¹⁰ VP/ml adenovirus (the expected upper safety limit) is only ˜7.5 μg/ml, which is unlikely to interfere with the AME testing. As shown in Table 6, A195 buffer containing preservatives were prepared and used in the antimicrobial effectiveness (AME) testing. Prior to the testing, sterile filtration using 0.22μ cellulose acetate membrane and/or one-week incubation at 37° C. was applied to selected samples as indicated in Table 6.

TABLE 6
HIV Vaccine Formulation Buffers with Preservatives

| | | | | 1 Week at |
|---|---|---|---|---|
| | | | Sterile | 37° C. |
| Preservative | Concentration | pH | Filtration | Incubation |
| A195 control | | 7.4 | Yes | No |
| Chlorobutanol | 0.25% (w/v) | 6.8 | Yes | No |
| Chlorobutanol | 0.40% (w/v) | 7.4 | No | No |
| Chlorobutanol | 0.50% (w/v) | 6.8 | Yes | Yes |
| Benzoic acid | 0.20% (w/v) | 6.8 | Yes | Yes |
| m-Cresol | 0.10% (v/v) | 7.4 | No | No |
| m-Cresol | 0.20% (v/v) | 6.8 | Yes | Yes |
| Methylparaben | 0.18% (w/v) | 7.4 | No | No |
| Propylparaben | 0.02% (w/v) | 7.4 | No | No |
| Methylparaben & | 0.20% (w/v) | 7.4 | Yes | No |
| Propylparaben | 0.02% (w/v) | | | |
| Methylparaben & | 0.18% (w/v) | 6.8 | Yes | Yes |
| Propylparaben | 0.02% (w/v) | | | |
| 2- | 0.40% (v/v) | 7.4 | No | No |
| Phenoxyethanol | | | | |
| 2- | 0.50% (v/v) | 6.8 | Yes | Yes |
| Phenoxyethanol | | | | |
| Benzyl alcohol | 1.00% (v/v) | 7.4 | Yes | No |
| Benzyl alcohol | 2.00% (v/v) | 7.4 | Yes | No |
| Phenol | 0.44% (v/v) | 7.4 | No | No |
| Benzethonium | 0.01% (w/v) | 6.8 | Yes | Yes |
| Cl | | | | |
| Benzalkonium | N/A | N/A | N/A | N/A |
| Cl | | | | |

The procedure of AME testing was described in the Materials and Methods section. The results are shown in the Table 7. Chlorobutanol, m-cresol, benzyl alcohol, phenol, and benzethonium chloride are effective antimicrobial preservatives when formulated in A195 buffer. For the chlorobutanol formulations: A195+0.4% CB passed USP criteria and A195+0.5% CB satisfied EP criteria B. However, another preservative compatible with Adeno stability, benzoic acid, failed AME testing. Benzoic acid is active in the nonionized (protonated) form. Therefore, the pKa of benzoic acid (4.2) limits its antimicrobial activity in A195 buffer (pH>6.4). Benzoic acid would not be expected to function effectively in A195, where the vast majority is in the ionized (unprotonated) state. Moreover, adenovirus would not be compatible with formulations having a pH near the pKa of benzoic acid.

TABLE 7
HIV Vaccine Formulation Buffers with Preservatives

| | | S. | P. | E. | C. | A. |
|---|---|---|---|---|---|---|
| Preservative | | aureus | aeruginosa | coli | albicans | niger |
| A195 | | Fail | Fail | Fail | USP | USP |
| CB | 0.25% | Fail | Fail | USP | EP-B | USP |
| | (w/v) | | | | | |
| CB | 0.40% | USP | USP | EP-B | EP-A | EP-B |
| | (w/v) | | | | | |
| CB | 0.50% | EP-B | EP-A | EP-B | EP-A | EP-B |
| | (w/v) | | | | | |
| BZ | 0.20% | Fail | Fail | Fail | Fail | USP |
| | (w/v) | | | | | |
| CR | 0.10% | USP | USP | USP | EP-B | EP-B |
| | (v/v) | | | | | |
| CR | 0.20% | EP-B | EP-B | USP | EP-A | EP-A |
| | (v/v) | | | | | |
| MP | 0.18% | USP | Fail | Fail | USP | USP |
| | (w/v) | | | | | |
| PP | 0.02% | Fail | Fail | Fail | USP | USP |
| | (w/v) | | | | | |
| MP & | 0.20% | USP | EP-A | EP-B | EP-A | EP-A |
| PP | (w/v) | | | | | |
| | 0.02% | | | | | |
| | (w/v) | | | | | |
| MP & | 0.18% | USP | EP-B | Fail | EP-B | EP-A |
| PP | (w/v) | | | | | |
| | 0.02% | | | | | |
| | (w/v) | | | | | |
| PE | 0.40% | USP | EP-B | Fail | EP-B | USP |
| | (v/v) | | | | | |
| PE | 0.50% | USP | EP-B | Fail | EP-B | USP |
| | (v/v) | | | | | |
| BA | 1.00% | EP-B | EP-A | EP-B | EP-A | EP-B |
| | (v/v) | | | | | |
| BA | 2.00% | EP-A | EP-A | EP-B | EP-A | EP-A |
| | (v/v) | | | | | |
| P | 0.44% | EP-B | EP-A | EP-B | EP-A | EP-A |
| | (v/v) | | | | | |
| BE | 0.01% | EP-A | EP-B | EP-B | EP-A | EP-B |
| | (w/v) | | | | | |
| BK | N/A | | | | | |

### EXAMPLE 3

### Immunogenicity Testing of HIV Vaccine in A195+Chlorobutanol

The AME data from Example 2 and the adenovirus stability data of Example show that chlorobutanol is a preferred preservative that is compatible with adenovirus stability that passed the USP and EP-B AME tests, when formulated in A195. To assess the potential effect of chlorobutanol on the vaccine potency, the immunogenicity of HIV vaccine formulated in A501 (A195 containing 0.4% chlorobutanol at pH 6.8) was tested in mice as described in Example 1 and in Table 7 below. Each formulation was used to vaccinate mice (10 mice/group) at dose 10⁹ VP/ml, 10⁸ VP/ml, and 10⁷ VP/ml, respectively. Immunogenicity of the vaccine was measured using ELISPOT and ELISA assays. The data in Table 8 show no significant difference in immune response to the vaccine with or without chlorobutanol.

TABLE 8
In Vivo Immunogenicity Result

| | | | | | | | ELISA | | |
|---|---|---|---|---|---|---|---|---|---|
| | | | | | | | (anti-HIV-1 P24 | | |
| | | | | ELISPOT | | | antibody titers) | | |
| | Dose | % | Mice/ | | Gag | | | SE | SE |
| Vector | (VP) | CB | Grp | Medium | 197-205 | p24 | GMT | upper | lower |
| MRKAd5gag | 1E9 | 0.40 | 10 | 1 | 456 | 132 | 135118 | 43171 | 32718 |
| MRKAd5gag | 1E9 | 0 | 10 | 2 | 656 | 207 | 89144 | 25187 | 19639 |
| MRKAd5gag | 1E8 | 0.40 | 10 | 7 | 858 | 308 | 19401 | 6199 | 4698 |
| MRKAd5gag | 1E8 | 0 | 10 | 10 | 796 | 301 | 67559 | 49387 | 28531 |
| MRKAd5gag | 1E7 | 0.40 | 10 | 4 | 544 | 287 | 1838 | 702 | 508 |
| MRKAd5gag | 1E7 | 0 | 10 | 28 | 826 | 205 | 1393 | 394 | 307 |
| None | none | | 10 | 15 | 4 | 49 | 50 | 0 | 0 |

### EXAMPLE 4

### Using Chlorobutanol in A195 for Adeno-Based HIV Vaccine

Live adenovirus formulated in A195 containing chlorobutanol was further characterized as described in this example section.

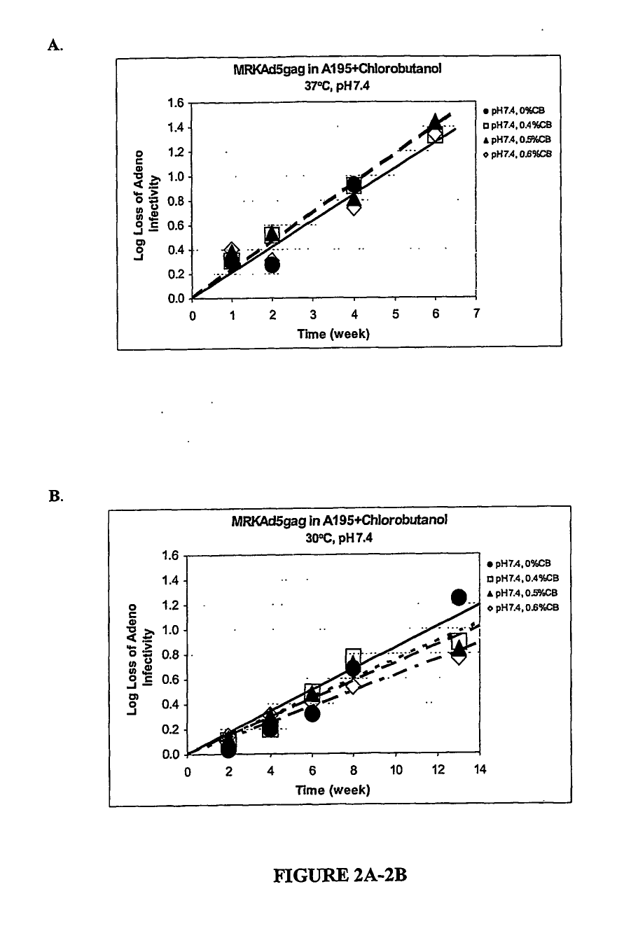

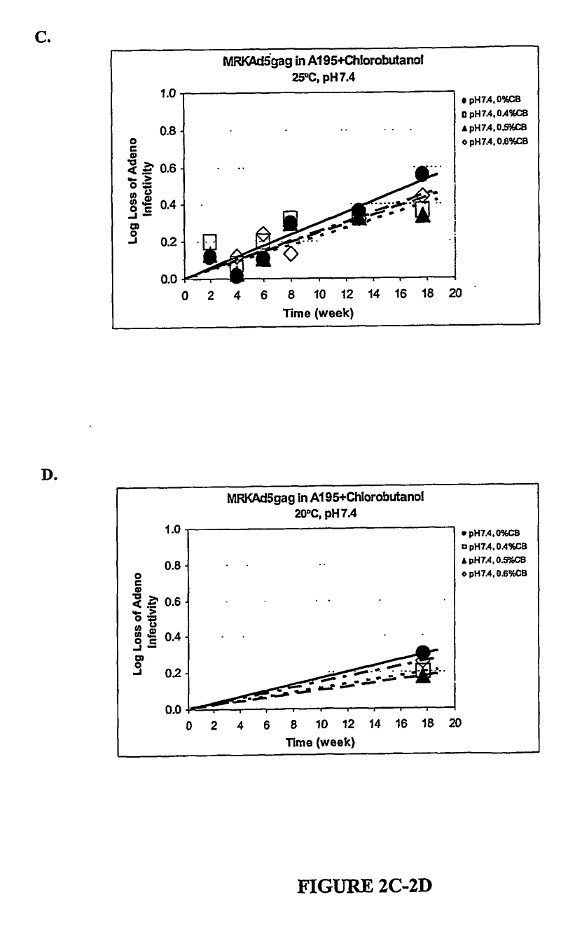

Chlorobutanol Concentration Effect—Adenovirus, MRKAd5gag, was formulated in A195 buffer containing 0.4%, 0.5%, and 0.6% (w/v) chlorobutanol and stored at 20, 25, 30, and 37° C. The remaining adenovirus infectivity was measured using the QPA assay and compared to corresponding control samples stored at −70° C. as described above. The results, as shown in FIG. 2A-D, indicate that CB at 0.4-0.6% in A195 did not significantly affect the stability of Ad5.

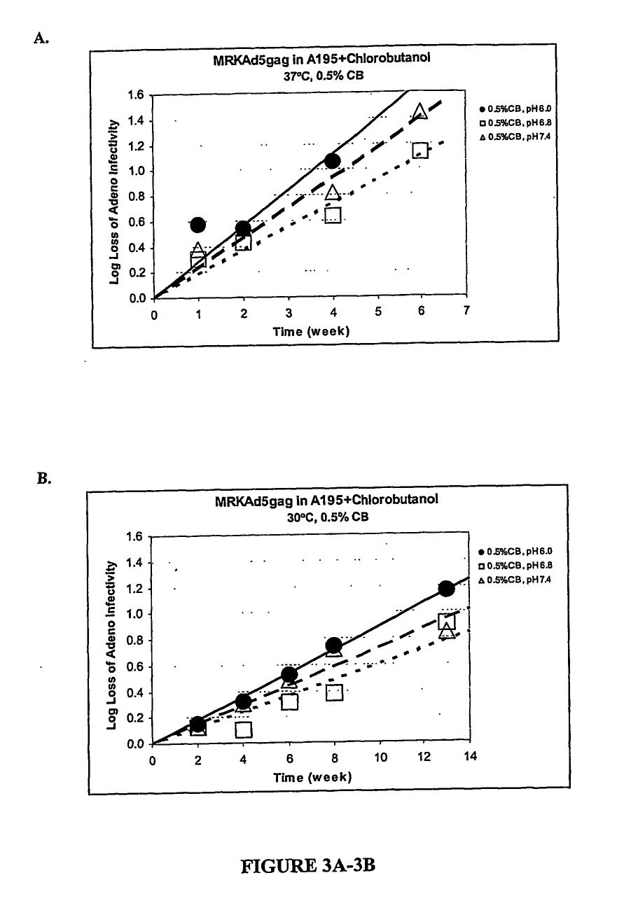

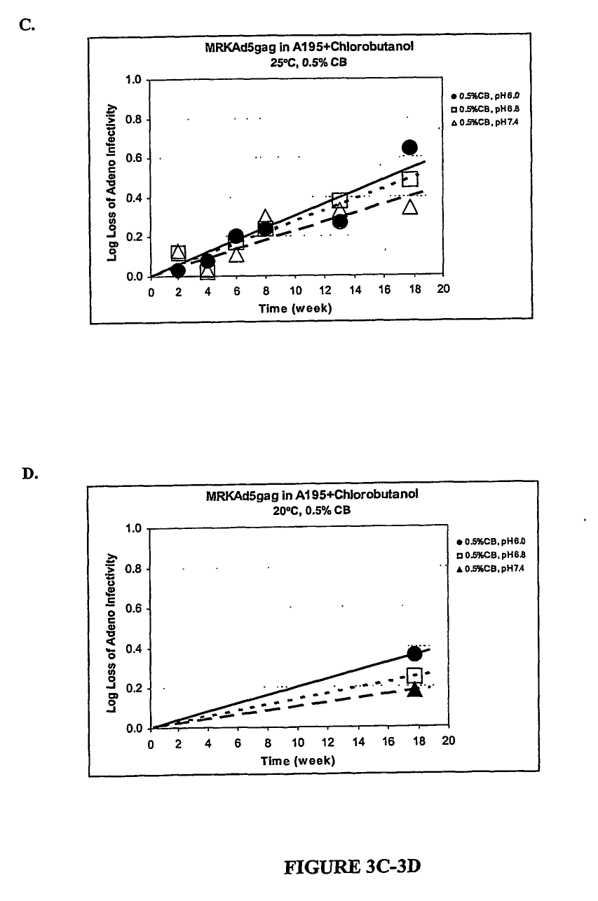

Optinum pH—MRKAd5gag was formulated in A195 buffer containing 0.5% (w/v) chlorobutanol at pH 6.0, 6,8 and 7.4, respectively and stored at 20, 25, 30, and 37° C. The remaining adenovirus infectivity was measured using the QPA assay and compared to corresponding control samples stored at −70° C. as described above. The data shown in FIG. 3A-D indicate similar stability at pH 6.8 and 7.4, but slightly lower stability at pH 6.0. An optimal pH range appears to be from about 6.8 to about 7.4. However, it will be within the purview of the artisan to determine an optimal pH range for the specific live adenovirus strain/vector for use in combination with chlorobutonal.

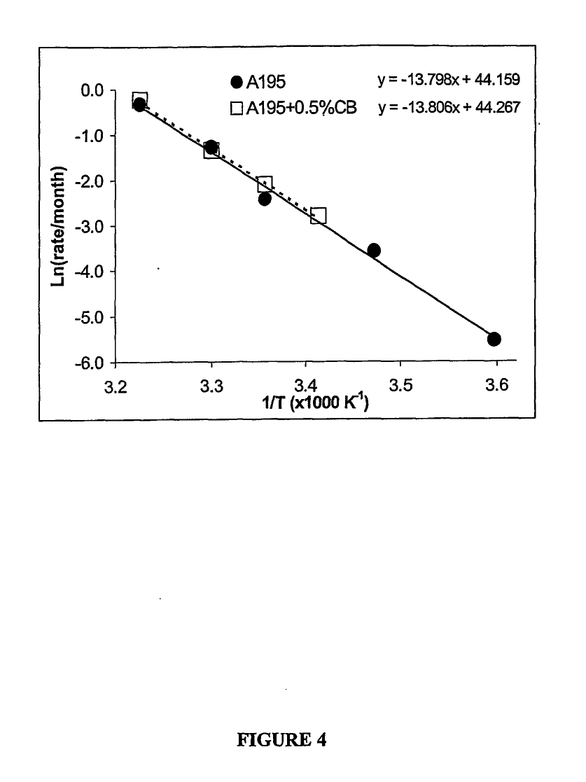

Long Term Stability of Adenovirus in A195+Chlorobutanol—The stability data of adenovirus in A195 (pH 6.8) containing 0.5% (w/v) chlorobutanol stored at 37, 30, 25, and 20° C. were collected and analyzed using the Arrhenius plot. The fitting overlaps with the stability data of Adeno in A195 collected previously. As shown in FIG. 4 the Arrhenius plot indicates that the projected loss of adenovirus infectivity in A195 is ≦0.1 logs after 2 years of 2-8° C. storage, with or without the presence of chlorobutanol. The data suggests that a long-term stability will be similar to the A195 formulation.

### EXAMPLE 5

### Stability of Chlorobutanol

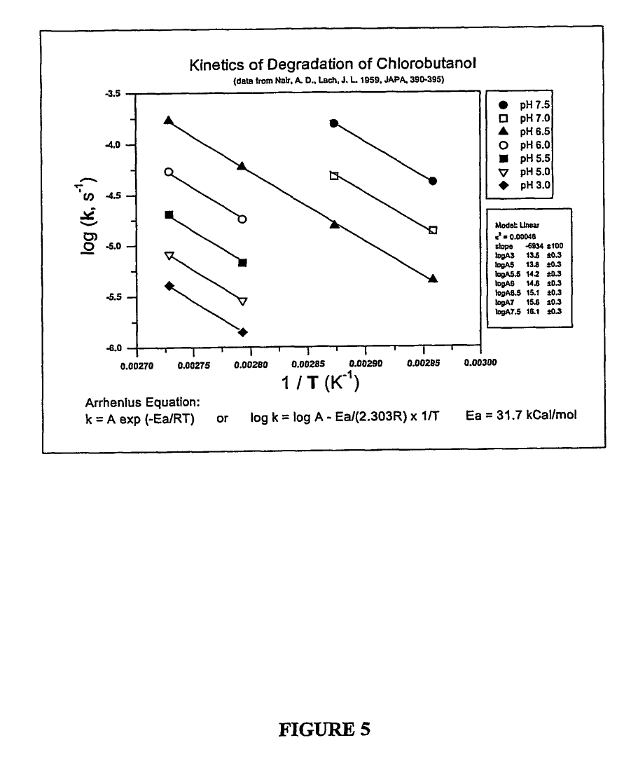

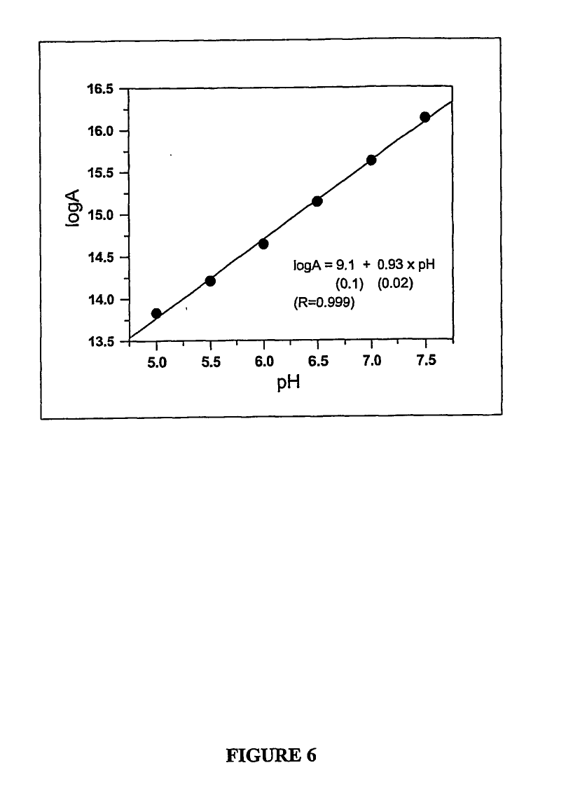

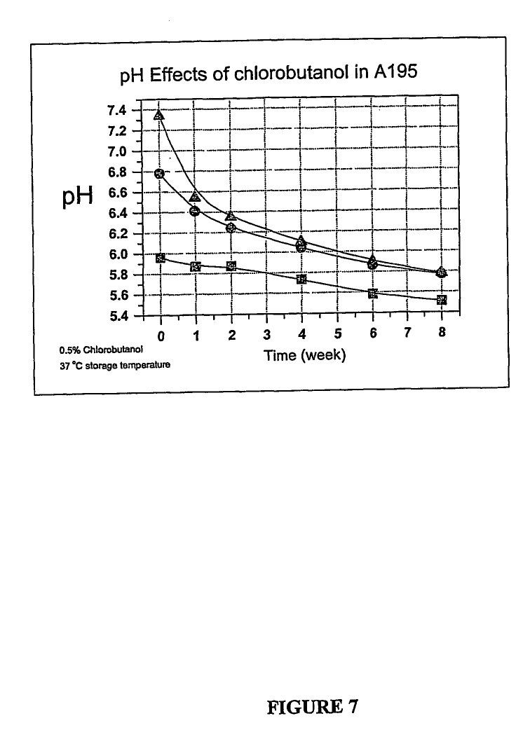

Chlorobutanol is not stable except under acidic conditions, which has curtailed its use. The degradation of chlorobutanol in aqueous solutions appears to be a specific hydroxide catalyzed reaction. The reaction is a pseudo first-order reaction with respect to chlorobutanol at pH 5.0-7.5 (Nair and, 1959, J. Am. Pharmaceutical Assoc. Vol. XLVIII, 390-395). Below is the mechanism of chlorobutanol degradation. The principal degradation products of chlorobutanol in aqueous solution were found to be acetone, carbon monoxide, H⁺, Cl⁻, and a trace amount of α-hydroxyisobutyric acid. Due to the production of H⁺ during hydrolysis, the pH of the solution may decrease during storage, depending on the buffer capacity of the formulation. 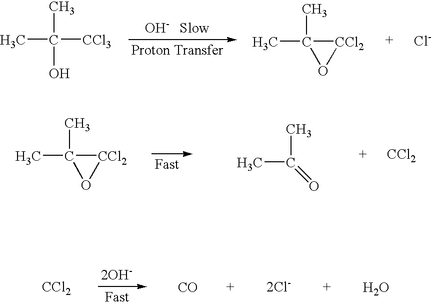
The degradation rate constants collected by Nair and Lach at different accelerated conditions were fitted simultaneously to the Arrhenius equation to obtain the apparent activation energy (Ea) for the degradation reaction and the Arrhenius factor A (FIG. 5). Ea was calculated to be 31.7 kCal/mole and the Arrhenius factors were listed in the figure and plotted in FIG. 6. Based on the fitted data in FIG. 5 and FIG. 6, the temperature and pH dependent degradation rate constant of chlorobutanol can be calculated. It is therefore predicted that at pH 6.8, 7.1% of the chlorobutanol will be degraded when stored at 37° C. for 1 week, and only 2.0% of the chlorobutanol will be degraded when stored at 5° C. for 2 years (shelf life). Therefore, the AME data after one-week of storage at 37° C. should be sufficient to cover the AME results at the end of vaccine shelf-life. The experimental data shown in FIG. 7 below: pH of A195+0.5% CB decreased from pH 6.8 to 6.4 after one week at 37° C.

The solubility of chlorobutanol in water at 20° C. is 0.8% (w/v) (Ref.: Authur H. Kibbe, 2000, Handbook of Pharmaceutical Excipients, 3rd Ed., pp 126-128). Therefore, the application of chlorobutanol as an antimicrobial preservative is primarily limited by its solubility. In the present invention chlorobutanol was used in aqueous formulations at 2-8° C. The concentration range of CB exemplified in the invention is 0.25-0.6% in A195 (pH 6.0 to 7.4). Because of CB at near saturation in aqueous buffers, the buffers were prepared by diluting a stock solution of CB in ethanol (48%, v/v) into A195 buffer (pH 6.0 to 7.4, no ethanol). Ethanol in the final solution helps to stabilize the solubility of CB in the aqueous buffers at exemplified concentrations. As shown in the Example 2, CB at 0.25% in A195 inhibits all microbials to different extends in the tests but based on the AME testing criteria, only testing for E. coli, C. albicans and A. niger passed USP or EP specifications. CB at 0.5% in A195 passed both USP and EP specifications for AME testing on all microbials tested. As shown in the Example 4, CB at concentration up to 0.6% has no significant effect on the stability of live adenovirus. Based on these results, a multi-dose adenovirus-based vaccine may be formulated in buffers containing various chlorobutanol concentrations, which show both antimicrobial activity and compatibility with adenovirus. This data is not presented to limit the present invention by disclosing any specific embodiment described herein. Indeed, various modifications of the invention in addition to those described herein will become apparent to those skilled in the art from the foregoing description. Such modifications are intended to fall within the scope of the appended claims.

What is claimed is:

1. A live adenovirus formulation comprising chlorobutanol.

2. A live adenovirus formulation of claim 1 wherein the formulation contains from a lowest effective concentration of chlorobutanol up to the solubility limit of chlorobutanol for said formulation.

3. A live adenovirus formulation of claim 1 wherein the formulation further comprises at least one inhibitor of free radical oxidation.

4. A live adenovirus formulation of claim 3 wherein the formulation further contains from a lowest effective concentration of chlorobutanol up to the solubility limit of chlorobutanol for said formulation.

5. A live adenovirus formulation of claim 3 wherein the inhibitor of free radical oxidation is selected from the group consisting of EDTA, ethanol, histidine, or combinations thereof.

6. A live adenovirus formulation of claim 5 wherein the formulation further contains from a lowest effective concentration of chlorobutanol up to the solubility limit of chlorobutanol for said formulation.

7. A live adenovirus formulation of claim 5 wherein the formulation further comprises a buffer, a cryoprotectant, a salt, a divalent cation, and a non-ionic detergent.

8. A live adenovirus formulation of claim 7 wherein the formulation further contains from a lowest effective concentration of chlorobutanol up to the solubility limit of chlorobutanol for said formulation.

9. A live adenovirus formulation of claim 1 with an adenovirus concentration in the range from about 1×10⁷ vp/mL to about 1×10¹³ vp/mL and a total osmolarity in a range from about 200 mOs/L to about 800 mOs/L.

10. A live adenovirus formulation of claim 9 wherein the formulation further contains from a lowest effective concentration of chlorobutanol up to the solubility limit of chlorobutanol for said formulation.

11. A live adenovirus formulation comprising chlorobutanol, wherein the formulation has been filled to present a multi-dose image.

12. A live adenovirus formulation of claim 11 wherein the formulation contains from a lowest effective concentration of chlorobutanol up to the solubility limit of chlorobutanol for said formulation.

13. A live adenovirus formulation of claim 11 wherein the formulation further comprises at least one inhibitor of free radical oxidation.

14. A live adenovirus formulation of claim 13 wherein the formulation further contains from a lowest effective concentration of chlorobutanol up to the solubility limit of chlorobutanol for said formulation.

15. A live adenovirus formulation of claim 13 wherein an inhibitor of free radical oxidation is selected from the group consisting of EDTA, ethanol, histidine, or combinations thereof.

16. A live adenovirus formulation of claim 15 wherein the formulation further contains from a lowest effective concentration of chlorobutanol up to the solubility limit of chlorobutanol for said formulation.

17. A live adenovirus formulation of claim 15 wherein the formulation further comprises a buffer, a cryoprotectant, a salt, a divalent cation, and a non-ionic detergent.

18. A live adenovirus formulation of claim 17 wherein the formulation further contains from a lowest effective concentration of chlorobutanol up to the solubility limit of chlorobutanol for said formulation.

19. A live adenovirus formulation of claim 11 with an adenovirus concentration in the range from about 1×10⁷ vp/mL to about 1×10¹³ vp/mL and a total osmolarity in a range from about 200 mOs/L to about 800 mOs/L.

20. A live adenovirus formulation of claim 19 wherein the formulation further contains from a lowest effective concentration of chlorobutanol up to the solubility limit of chlorobutanol for said formulation.

21. A filled multi-dose vaccine vial comprising live adenovirus and chlorobutanol.

22. The multi-dose vaccine vial of claim 21 wherein the formulation contains from a lowest effective concentration of chlorobutanol up to the solubility limit of chlorobutanol for said formulation.

23. The multi-dose vaccine vial of claim 21 wherein the formulation further comprises at least one inhibitor of free radical oxidation.

24. The multi-dose vaccine vial of claim 23 wherein the formulation further contains from a lowest effective concentration of chlorobutanol up to the solubility limit of chlorobutanol for said formulation.

25. The multi-dose vaccine vial of claim 23 wherein an inhibitor of free radical oxidation is is selected from the group consisting of EDTA, ethanol, histidine, or combinations thereof.

26. The multi-dose vaccine vial of claim 25 wherein the formulation further contains from a lowest effective concentration of chlorobutanol up to the solubility limit of chlorobutanol for said formulation.

27. The multi-dose vaccine vial of claim 25 wherein the formulation further comprises a buffer, a cryoprotectant, a salt, a divalent cation, and a non-ionic detergent.

28. The multi-dose vaccine vial of claim 27 wherein the formulation further contains from a lowest effective concentration of chlorobutanol up to the solubility limit of chlorobutanol for said formulation.

29. The multi-dose vaccine vial of claim 21 with an adenovirus concentration in the range from about 1×10⁷ vp/mL to about 1×10¹³ vp/mL and a total osmolarity in a range from about 200 mOs/L to about 800 mOs/L.

30. The multi-dose vaccine vial of claim 29 wherein the formulation further contains from a lowest effective concentration of chlorobutanol up to the solubility limit of chlorobutanol for said formulation.

31. A method of preserving a live adenovirus formulation which comprises adding chlorobutanol to the formulation, such that addition of chlorobutanol maintains adequate antimicrobial effectiveness while maintaining stability of the adenovirus for at least one year when stored at 2-8° C.

32. The method of claim 31 wherein the formulation contains from a lowest effective concentration of chlorobutanol up to the solubility limit of chlorobutanol for said formulation.

33. The method of claim 31 wherein the formulation is filled as a single dose image.

34. The method of claim 31 wherein the formulation contains from a lowest effective concentration of chlorobutanol up to the solubility limit of chlorobutanol for said formulation.

35. The method of claim 31 wherein the formulation is filled as a multi-dose image.

36. The method of claim 35 wherein the formulation contains from a lowest effective concentration of chlorobutanol up to the solubility limit of chlorobutanol for said formulation.
